(19) United States
(12) Patent Application Publication
(10) Pub. No.: US 2005/0176623 A1
(43) Pub. Date: Aug. 11, 2005

(54) Non-invasive treatment of disease using amphipathic compounds

(76) Inventor: Neil Wagle, Brookline, MA (US)

Correspondence Address: BOURQUE & ASSOCIATES, P.A., 835 HANOVER STREET, SUITE 303, MANCHESTER, NH, 03104, US

(21) Appl. No.: 11/057,091

(22) Filed: Feb. 11, 2005

Related U.S. Application Data
(60) Provisional application No. 60/543,124, filed on Feb. 11, 2004.

(51) Int. Cl. A61K 38/17

(52) U.S. Cl. 514/2

(57) ABSTRACT

The present invention features a non-invasive system and method for delivering apolipoprotein, amphipathic compounds, and the like into the blood stream using pulmonary delivering. Apolipoprotein, amphipathic compounds, and the like is suspended in a solvent, preferably a saline solution. Next, the soluation is nebulized to form a plurality of droplets sized to reach the periphery of the lung. Once proximate the lung, the apolipoprotein, amphipathic compounds, and the like is dissolved through the lung interface and into the serum. Due to its physical and chemical properties, apolipoprotein A-I is effectively absorbed into the serum through the lung. The present invention can be used for the treatment of cardiovascular disease as well Alzheimer's Disease and other.

### CROSS-REFERENCE TO RELATED APPLICATIONS

This application claims the benefit of U.S. Provisional Application No. 60\543,124, filed Feb. 11, 2004.

### TECHNICAL FIELD

The present invention relates to coronary heart disease and more particularly, relates to apparatus and methods for non-invasively treating coronary heart disease.

### BACKGROUND INFORMATION

Coronary heart disease (CHD) has been at epidemic proportions for over half a century. Current data suggest that 13.2 million adults (6.4%) in the United States have CHD, including 7.8 million who have suffered myocardial infarctions (MI) and 6.8 million who have angina pectoris. As adults reach the age of 40, the lifetime risk of developing CHD is 49% in men and 32% in women. This extraordinarily high prevalence has made CHD the leading cause of death in adults in the United States, accounting for nearly one third of deaths in individuals over age 35. The direct and indirect cost to society for the treatment of this disease has been estimated at $133.2 billion dollars, making CHD the most expensive disease in the U.S. in addition to the most prevalent.

Atherosclerosis is considered to be responsible for almost all cases of CHD. Although CHD is the most prevalent clinical manifestation of atherosclerosis, it is by no means the only one. Other clinical manifestations of atherosclerosis include cerebrovascular disease, peripheral vascular disease, and renal artery stenosis, all of which are highly prevalent cardiovascular diseases. Together, atherosclerotic cardiovascular diseases are the cause of mortality of 50% of the populations of the United States, Europe, and Japan.

Extensive research has been conducted to determine the pathophysiology of atherosclerosis. At the most basic level, atherosclerosis (FIG. 1) is caused by high concentrations of certain types of lipoproteins, namely low density lipoproteins (LDL) and very low density lipoproteins (VLDL). High levels of LDL and VLDL in the blood stream cannot be completely cleared by cells in the body. As a result, excess LDL remains in circulation, ultimately becoming oxidized. Oxidized LDL deposits beneath endothelial cells in blood vessels and accumulates under the tunica intima, the innermost layer of the blood vessel wall. After the deposition of oxidized LDL, macrophages and T cells extravasate into the sub-intimal layer in an attempt to remove the deposits. The result is the release of many inflammatory cytokines that ultimately leads to large collections of cholesterol-filled macrophages known as “foam” cells. These inflamed collections of foam cells are called atheromas, the defining feature of atherosclerosis. Atheromas cause the diseases described above in two primary ways (FIG. 1).

First, atheromas may narrow the lumen of the blood vessel as they grow. As a result, the tissues the vessel serves are subject to ischemic damage. Furthermore, an embolus traveling in the blood can easily lodge in these narrow areas, infarcting the downstream tissue.

Secondly, atheromas themselves can rupture. The spilling of foam cells and cholesterol into the vessel can occlude the artery, causing an infarction, or the debris can become a dangerous embolus itself. Alternatively, the rupture can “heal”, resulting in further occlusion of the lumen of the artery. By these mechanisms, atherosclerosis causes cardiovascular disease, most commonly CHD, cerebrovascular accidents, peripheral vascular disease, and renal artery stenosis.

The medical community has long recognized the seriousness of atherosclerosis and cardiovascular disease. The result has been widespread use of preventative measures such as dietary changes aimed at reducing the intake of cholesterol, and medical therapies such as the administration of HMG CoA reductase inhibitors (statins) aimed at upregulating LDL receptors by reducing the amount of cholesterol the body produces. Statins have been widely successful in lowering LDL; in fact, Lipitor (Pfizer) and Zocor (Merck) are currently two of the highest grossing medications in the U.S.

Despite the effectiveness of statins at reducing serum LDL levels, the incidence of cardiovascular disease has not declined as much as expected. While decreasing LDL levels impedes the progression of atherosclerosis, it does not cause the regression of existing atherosclerosis. Since individuals are not placed on statins until they are identified as having a higher than average risk of cardiovascular disease, nearly all have a significant degree of atherosclerosis by the time they begin therapy. Even individuals who are not at a high risk of cardiovascular complications develop significant atherosclerosis by mid-life. Fatty streaks in blood vessels begin to develop during the early 20's in most individuals; by later life, these can progress into significant atherosclerosis for even lower risk individuals. Due to the ubiquitous nature of atherosclerosis and the absence of a treatment that consistently and effectively reverses atherosclerosis, a clear necessity for a novel therapy exists.

The only known way to reverse atherosclerosis is through high density lipoprotein (HDL)-mediated reverse cholesterol transport. It has long been known that HDL levels are inversely proportional to the degree of atherosclerosis in a patient. Significant evidence suggests that this is primarily due to reverse cholesterol transport from the cholesterol-filled foamy macrophages that compose atheromas (FIG. 2), the mechanism of which has been elucidated in recent decades.

Nascent high-density lipoprotein (HDL) is discoidal initially and is composed of a lipid (phosphotidylcholine) bilayer studded with two to four molecules of apolipoprotein A-I (apoA-I), the defining protein of HDL. Circulating nascent HDL interacts with peripheral cells, most relevantly with foam cells. These foam cells generally hydrolyze some of their cholesterol esters into free cholesterol in their cytoplasm.

Mediated by apoA-I, the ATP-binding cassette transporter A-1 (ABCA1) in the membrane of the foamy macrophages transfers the free cholesterol from the macrophage to the phospholipid bilayer of the nascent HDL.

Once cholesterol is transferred to the HDL membrane, apoA-I activates an enzyme called lecithin cholesterol acyl transferase (LCAT) that esterifies the cholesterol from the transmembrane form to a form that associates with the hydrophobic tails of the membrane phospholipids. The initially discoidal nascent HDL is now “inflated” into a spherical mature HDL. After being filled with cholesterol from peripheral cells, the mature HDL returns to circulation. ApoA-I then activates a liver receptor, scavenger receptor B-I (SR-BI), which accepts the cholesterol esters from the HDL and excretes them in the bile. In this way, HDL is able to transport cholesterol out of the foamy macrophages that compose atheromas, thereby reducing the degree of atherosclerotic plaque.

In addition to reverse cholesterol transport, two other protective mechanisms of HDL have been proposed. The first is that HDL protects LDL from oxidation, eliminating the first step in atherogenesis. The second is that the presence of HDL selectively downregulates the cell adhesion molecules (CAMs) that facilitate the extravasation of inflammatory immune cells, eliminating the second step of atherogenesis. In addition to the ability of HDL to cause atheromas to regress through reverse cholesterol transport, these protective features of HDL limit atherogenesis, making HDL therapies even more attractive.

The ability of HDL therapy to induce the regression of atherosclerosis has been confirmed in animals multiple times since the early 1990's. Badimon, J. J., et al, demonstrated in 1990 that atherosclerotic lesions regressed after the infusion of HDL into rabbits on a high cholesterol diet. See Badimon, J. J., Badimon, L., Fuster, V. Regression of atherosclerotic lesions by high density lipoprotein plasma fraction in the cholesterol-fed rabbit. Journal of Clinical Investigation. 1990; 85:1234-1241, fully incorporated herein by reference.

Transgenic mice engineered to overexpress apoA-I were shown to have higher levels of HDL and to be resistant to atherogenesis both from genetically induced means and from diet induced means. See Plump, A. S., Scott, C. J., Breslow, J. L. Human apolipoprotein A-I gene expression increases high density lipoprotein and suppresses atherosclerosis in the apolipoprotein E-deficient mouse. Proceedings of the National Academy of Sciences, USA. 1994; 91:9607-9611; and 13 Rubin, E. M., Krauss, R. M., Spangler, E. A., Verstuyft, J. G., Cliff, S. M. Inhibition of early atherogenesis in transgenic mice by human apolipoprotein A-I. Nature. 1991; 353:265-267, both fully incorporated herein by reference.

These experiments were critical to the recognition of HDL, and more specifically apoA-I, as a potential therapeutic target for the treatment of atherosclerosis.

Recently, HDL therapy has progressed from animals to humans for the first time. A recent paper in the Journal of the American Medical Association describes an isoform of apoA-I (apoA-I Milano) found in a small population in Italy that confers some degree of protection from atherosclerosis. See Nissen, S. E., Tsunoda, T., Tuzcu, E. M., Schoenhagen, P., Cooper, C. J., Yasin, M., Eaton, G. M., Lauer, M. A., Sheldon, S., Grines, C. L., Halpern, S., Crowe, T., Blankenship, J. C., Kerensky, R. Effect of recombinant apoA-I Milano on coronary atherosclerosis in patients with acute coronary syndromes: a randomized controlled trail. Journal of the American Medical Association. 2003; 290 (23):712-719, fully incorporated herein by reference.

Synthetic apoA-I Milano liposomes were injected weekly for five weeks at concentrations of 0 mg/kg (control), 15 mg/kg (low dose), or 45 mg/kg (high dose) in 47 patients who had suffered an acute coronary syndrome less than 14 days prior to the start of the study. Patients who received the treatment (either dose) had a mean reduction in atheroma volume of 1.06% versus controls who had a mean increase in volume of 0.14%. Given these results, the potential of this new therapy was evident. Despite the fact that Esperion, the proprietary owner of apoA-I Milano, did not have a product on the market, the company was purchased for $1.3 billion dollars just one month after the publication of these data.

Interestingly, the ability of wild-type apoA-I to reduce atherosclerosis has never been tested in humans. It has been suggested that apoA-I Milano may not be more effective than wild-type apoA-I at reverse cholesterol transport. See Rader, D. J. High-Density lipoproteins as an emerging therapeutic target for atherosclerosis. Journal of the American Medical Association. 2003; 290 (17):2322-2324, fully incorporated herein by reference. However, in none of its sponsored studies has Esperion ever tested the relative efficacy of apoA-I Milano against wild-type apoA-I.

In addition to injecting HDL, there are a number of methods of promoting reverse cholesterol transport currently under investigation. Some statins have been shown to raise serum HDL levels 10% to 15%. See Brewer, H. B., Jr. Benefit-risk assessment of Rosuvastatin 10 to 40 milligrams. American Journal of Cardiology. 2003; 92:23K-29K, fully incorporated herein by reference. Additionally, fibrates and niacin have been shown to increase HDL levels 25% to 30%. See Rader, D. J. Effects of nonstatin lipid drug therapy on high-density lipoprotein metabolism. American Journal of Cardiology. 2003; 91:18-23, fully incorporated herein by reference.

Recently, the most attention has been given to cholesterol ester transfer protein inhibitors (CETP-i). CETP mediates the transfer of cholesterol esters from HDL to VLDL and LDL; it is hypothesized that it therefore reduces serum HDL levels and increases serum LDL levels. Consequently, many believe that a CETP inhibitor would raise HDL and lower LDL. Although there is still a considerable degree of controversy as to the ability of each of these therapies to successfully decrease extant atherosclerosis, the intensity with which such a therapy is being sought is testament to the medical need.

Given the current lack of an effective method of reversing atherosclerosis combined with the decades of research suggesting that apoA-I administration would perform precisely this function, the need to explore apoA-I therapy is evident. The administration of apoA-I could markedly improve the prognosis for the hundreds of millions of people with atherosclerosis-induced pathologies. However, one sizable hurdle remains for the therapy: delivery.

Intravenous therapies, including the aforementioned ApoA-I Milano therapy, are difficult to sustain. The therapy must be administered by a trained professional and requires outpatient visits, waiting time, administration time and personnel, and high cost to the medical system. The high cost per patient combined with the high prevalence of the condition raises costs for insurance companies, which are then passed on to the patients paying the premium.

From the perspective of the patient, invasive therapies are often associated with a reluctance to begin therapy and noncompliance once therapy begins. Due to the high cost and the inconvenience of treatment, intravenous injection of apoA-I or any other injectable HDL therapy would be reserved for the most severe cases. Millions of individuals with cardiovascular disease would not receive life-saving treatment.

Accordingly, what is needed is an apparatus and method for non-invasively treating coronary heart disease. The method and apparatus preferably delivers a biologically active compound to the lungs and should preferably be readily absorbed into the blood stream. What is also needed is a non-invasive method and apparatus which leads to increased HDL concentration and ultimately to reduced atherosclerosis.

What is further needed is a method and apparatus is a method of delivering apoA-I (or a related or derivative compound) such that delivery via the lungs is possible. Delivery via the lungs requires that the formulation be nebulizable to a droplet size that will reach the peripheral lung and that these small droplets contain apoA-I (or a related or derivative compound).

The apparatus and method should also preferably include a formulation of apoA-I that will allow for significant absorption across the alveolar exchange surface.

It is important to note that the present invention is not intended to be limited to a system or method which must satisfy one or more of any stated objects or features of the invention. It is also important to note that the present invention is not limited to the preferred, exemplary, or primary embodiment(s) described herein. Modifications and substitutions by one of ordinary skill in the art are considered to be within the scope of the present invention, which is not to be limited except by the following claims.

### SUMMARY

According to one embodiment, the present invention features a method for treating a medical condition including the act of non-invasively administering a medicament comprising at least one amphipathic compound. The act of non-invasively administering is preferably selected from the group consisting of the acts of pulmonarily administering, transdermally administering, nasally administering, sublingually administering, and ocularly administering. The amphipathic compound preferably includes at least one apolipoprotein such as, but not limited to, at least one apolipoprotein chosen from the group consisting of wild-type apolipoprotein A-I, synthetic apolipoprotein A-I, apolipoprotein E, apolipoprotein E-2, and apolipoprotein J.

The method optionally includes the act of preparing the amphipathic compound in a solvent. The solvent preferably includes at least one solvent chosen from the group consisting of saline, surfactant/phospholipids, benzalkonium chloride, calcium chloride, and sodium citrate. Optionally, the amphipathic compound is added at a low pH and subsequently mixing vigorously while rapidly raising the pH past the isoelectric point of the amphipathic compound.

The method may also include the act of transporting the at least one apolipoprotein, preferably apolipoprotein A-I (apoA-I) or apolipoprotein E (apoE), into a blood stream to effect therapies directed toward decreasing the extent of atherosclerosis. In the preferred embodiment, approximately 150 mg of apoA-I is transported into the blood stream daily. The apoA-I may include substantially only the helical portion of apoA-I.

The act of non-invasively administering may include pulmonarily administering an effective amount of the amphipathic compound, wherein the amphipathic compound includes an apolipoprotein (preferably an amphipathic compound less than 200 kDa in size) prepared in a solvent. Again, the solvent is preferably chosen from the group consisting of saline, surfactant, benzalkonium chloride, calcium chloride, and sodium citrate.

The act of pulmonarily administering the apolipoprotein preferably includes forming a substantially heterodispersed aerosol having a hydrodynamic radius of approximately 2 to approximately 5 um.

According to another embodiment, the present invention features a non-invasive method of delivering amphipathic compound comprising the acts of preparing a composition containing at least one amphipathic compound; aerosolizing the composition to create a plurality of droplets, wherein the plurality of droplets include the at least one amphipathic compound; and delivering the aerosolized composition to a periphery of a lung, wherein the at least amphipathic compound is transported through an interface of a lung into the blood stream.

In the preferred embodiment, the aerosolizing of the composition is accomplished by a device chosen from the following group consisting of a jet nebulizer, an ultrasonic nebulizer, a dry powder inhaler, a liquid inhaler, a metered dose inhaler, or any device that creates an aerosol with inhalable droplets containing the amphipathic compound.

The act of aerosolizing the composition preferably includes forming a substantially heterodispersed aerosol having a hydrodynamic radius of approximately 2 to approximately 5 um and also preferably includes forming a substantially even monolayer of the composition proximate the interface of the lung.

In the preferred embodiment, the method optionally includes the act of preparing the composition further includes combing the at least one amphipathic compound in a solvent. The solvent is preferably chosen from the group consisting of saline, surfactant, benzalkonium chloride, calcium chloride, and sodium citrate. The amphipathic compound is preferably added at a low pH and subsequently mixing vigorously while rapidly raising the pH past the isoelectric point of the amphipathic compound.

The amphipathic compound preferably is less than 200 kDa in size and preferably includes at least one apolipoprotein. The apolipoprotein is preferably chosen from the group consisting of wild-type apolipoprotein A-I, synthetic apolipoprotein A-I, apolipoprotein E, apolipoprotein E-2, and apolipoprotein J.

According to yet another embodiment, the present invention features a device for non-invasively delivering one or more amphipathic compounds into a blood stream. The device includes a reservoir containing a solution of at least one amphipathic compound in a solvent. A metering device is in fluid communication with the reservoir and measures a quantity of the solution to be administered. An aerosolizing device creates a plurality of droplets sized to be deliver the aerosolized solution to a periphery of a lung.

The amphipathic compound preferably includes at least one apolipoprotein chosen from the group consisting of wild-type apolipoprotein A-I, synthetic apolipoprotein A-I, apolipoprotein E, apolipoprotein E-2, and apolipoprotein J. The solvent preferably includes at least one solvent chosen from the group consisting of saline, surfactant, benzalkonium chloride, calcium chloride, and sodium citrate.

### BRIEF DESCRIPTION OF THE DRAWINGS

These and other features and advantages of the present invention will be better understood by reading the following detailed description, taken together with the drawings wherein:

FIG. 1 is an illustrative embodiment showing the effects of cardiovascular disease;

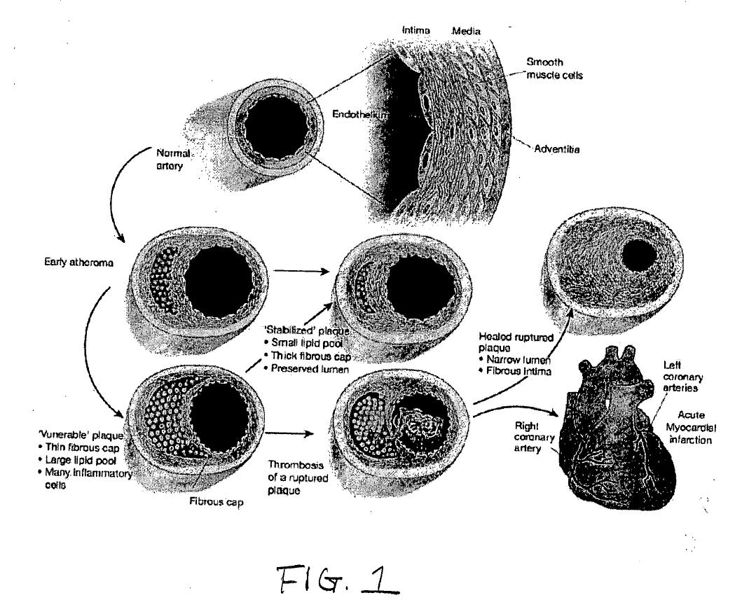

FIG. 2 is an illustrative embodiment showing reverse cholesterol transport;

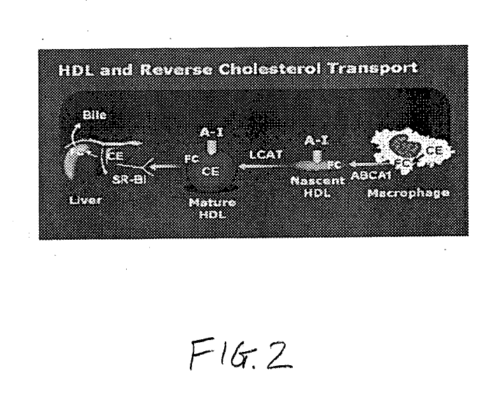

FIG. 3 is an illustrative embodiment showing the barriers to absorption;

FIG. 4 is a chart showing the effect of delivered ApoA-I on serum HDL levels in humans; and

FIG. 5 is a block diagram of one embodiment of the apparatus for the non-invasive delivery of apolipoproteins, amphipathic compounds, and the like to the periphery of the lung.

### DETAILED DESCRIPTION OF THE PREFERRED EMBODIMENTS

According to one embodiment, the present invention features a non-invasive delivery system and method for treating diseases such as, but not limited to, cardiovascular diseases. In the preferred embodiment, the present invention features a system and method for pulmonary delivery of one or more therapeutic agents to treat cardiovascular disease through reverse cholesterol transport.

As used in the present application, the term “non-invasive” is intended to denote a delivery method and/or apparatus that does not involve the puncturing or incision of the skin or a membrane or the insertion of an instrument into the body. For purposes of this application, the term non-invasive is not intended to include enteral delivery. For illustrative purposes only, the present invention will be described wherein the non-invasive delivery method and apparatus includes a pulmonary delivery method and apparatus, though one skilled in the art will recognize that the present invention also includes nasal, sublingual, transdermal, and transocular methods and apparatus.

For illustrative purposes only, the present invention will be described wherein the therapeutic compound includes apolipolprotein, and specifically apolipoprotein A-1 (ApoA-1, either synthetic or wild-type apoA-I). Those skilled in the art will understand that other therapeutic compounds are also within the scope of the present invention such as, but not limited to, other amphipathetic compounds such as, but not limted to apolipoproteins including, but not limited to, apolipoprotein E, apolipoprotein E-2, or apolipoprotein J. Additionally, those skilled in the art will understand that the present invention can also be used for treating other diseases and conditions. For example, the present invention may be used with ApoE-2 for treating Alzheimer's.

As mentioned, the preferred embodiment of the present invention utilizes pulmonary delivery. While pulmonary delivery has long been used for the treatment of pulmonary diseases, it is only recently that the lung has been explored as a route of administration for the treatment of systemic diseases. Besides being non-invasive, the lungs have both an enormous absorptive potential and a low rate of clearance, allowing therapeutic agents to enter the systemic circulation. The absorptive potential of the lungs comes from their surface area, which is approximately 100 m², their highly permeable membrane, which is approximately 0.2 to approximately 0.7 μm in thickness and contains peptide transporters, and their dense vasculature, since the peripheral lungs are the only organs in the body to receive nearly the entire cardiac output.

The low clearance of therapeutic agents in the peripheral lung is due to a low enzymatic environment and slow mucociliary clearance outside of the conducting airways. Moreover, agents absorbed in the lung avoid dietary and digestive complications and are not cleared by hepatic or renal first pass. Tests demonstrate that most proteins delivered to the alveolar epithelium are absorbed by passive diffusion into the blood stream without degradation, leaving proteins intact in the serum.

Because of several perceived problems, drawbacks, and limitations, however, pulmonary delivery has heretofore not realized the pervasiveness anticipated by the scientific community.

One of the main perceived problems that has heretofore prevented or otherwise diverted those skilled in the art from exploring pulmonary delivery is lack of absorption. Because only the alveolar interface is ideal for delivery, any materials trapped in the oropharynx or the conducting airways do not absorb well and are mainly digested. For materials that do reach the alveolar interface, certain formulations are not suitable for absorption. The size, hydrophilicity, charge, or other physical characteristics often prevent the absorption of materials into pneumocytes.

In addition to this lack of absorption, another commonly perceived problem that has heretofore prevented or otherwise diverted those skilled in the art from exploring pulmonary delivery is toxicity. The low rate of clearance (discussed above) as one of the advantages of pulmonary delivery is also one of its biggest disadvantages. Some formulations of certain materials deposited in the peripheral lung that are not cleared have been claimed to cause inflammation and possibly fibrosis. This kind of toxicity would preclude the use of pulmonary delivery.

While most research in the field of pulmonary delivery is currently seeking absorption enhancers and nontoxic formulations, the inventor of the present application has discovered that the physiochemical properties of apoA-I make such measures unnecessary. In fact, the inventor of the present application has discovered that the chemical and physical properties of apoA-I make it an ideal candidate for pulmonary delivery, allowing it to circumvent the problems discussed above. Both of these discoveries will be discussed in greater detail hereinbelow.

The inventor has discovered that the size of apoA-I is suitable for absorption across the lung epithelium. ApoA-I is a relatively small protein (26 kDa) and tests indicate that the lung epithelial barrier only effectively restricts the absorption of species greater than 200 kDa.

In contrast to materials that aggregate or precipitate when delivered to the lung, the inventor has also discovered that apoA-I administered to the peripheral lung is efficiently absorbed. ApoA-I is an amphipathic protein, meaning that it has both a hydrophilic and a hydrophobic domain. This property causes apoA-I to spontaneously associate with phospholipids. This is ideal for pulmonary delivery because the alveolar interface is coated with surfactant, composed of phospholipids and proteins. As a result, apoA-I administered to the peripheral lung will spontaneously and evenly disperse across the surfactant-coated surface and is then be distributed to facilitate efficient absorption.

The inventor has discovered that even distribution of apoA-I in the surfactant aids in its absorption in two ways. The surfactant is regularly turned over and absorbed by pneumocytes. The present application includes the realization that the associated apoA-I will be absorbed with it. In addition to this mechanism of transport, the even distribution along the surface of the interface allows apoA-I to have access to the two primary mechanisms of absorption, namely paracellular transport and transporter-mediated transcytosis performed by transporters such as PEPT1, PEPT2, and others.

The inventor of the present invention has analyzed the properties of the alveolar exchange surface and the properties of apoA-I to determine that apoA-I should readily be absorbed in the lung. Existing tests have shown that proteins reaching the alveolar surface are primarily cleared from the lung by passive, non-degradative absorption into the blood stream. As a result, apoA-I delivered to the lung will enter the serum intact.

In addition to this general property of the lung epithelium, the inventor has discovered that apoA-I is specifically suited to pulmonary delivery. One factor is that ApoA-I is a relatively small protein with a molecular mass of 26 kDa and a hydrodynamic radius of 3.5 to 6.8 nm. Tests show that the lung epithelial barrier only effectively restricts the passage of proteins larger than 200 kDa. Accordingly, apoA-I should absorb efficiently.

Perhaps even more significant than its small size, apoA-I is an amphipathic protein, meaning that it has both a hydrophobic domain and a hydrophilic domain. This is the property of apoA-I that allows it to spontaneously associate with phospholipids. Phospholipids, primarily dipalmitoylphosphotidylcholine (DPPC), and small amounts of protein are the primary components of the surfactant that covers the epithelial surface of the lung. The inventor has discovered that the physical chemistry of apoA-I will most likely cause any apoA-I reaching the lungs to spontaneously disperse and integrate into the surfactant monolayer. The present invention features the realization that these are the ideal conditions for pulmonary delivery.

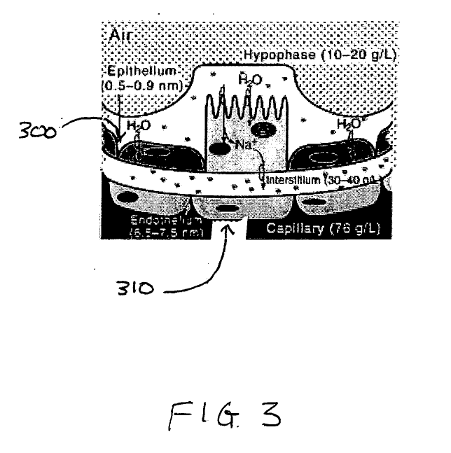

Once the apoA-I has integrated into the surface of the monolayer, the inventor of the present application has discovered that apoA-I will be readily absorbed into the blood stream. Two barriers for particles in the air space to pass through exist, namely, the epithelial cell layer 300, FIG. 3, and the endothelial layer 310. Absorption of therapeutic agents from the epithelium 300 primarily occurs through two processes: paracellular transport and transporter-mediated transcellular transport.

Paracellular transport in the lungs occurs through gaps between pneumocytes and is dependent on the gradient between materials on either side. The transporters for proteins are PEPT1 and PEPT2; these and other transporters have been shown to transport materials from the surfactant layer into pneumocytes. The mechanisms by which materials in pneumocytes are transported into the interstitium and subsequently into the serum are currently unknown, but may involve constitutive transporters on the basolateral surface of epithelial cells. Due to the much larger effective pore size of the endothelial cell layer 310, the epithelial cell layer 300 is the limiting step for absorption. The rates of both mechanisms of transport through the epithelial cell layer 300 are determined by the surface contact of the therapeutic agent on the absorptive surface.

Perhaps even more beneficial than the absorptive properties of apoA-I is the potential lack of toxicity. Most of the inflammation and fibrosis that occurs when proteins are delivered to the lung occur because of unabsorbed, uncleared aggregates of protinacious material residing in the alveolar space. The inventor of the present application has discovered that the physiochemical behavior of apoA-I with phospholipids, namely the dispersal of apoA-I into an even monolayer, prevents any protein aggregates or solubility problems in the lung that might lead to inflammation and fibrosis. Moreover, rapid absorption of apoA-I further reduces the residence time of apoA-I in the lung.

In addition to the beneficial properties that apoA-I possesses, it is important to note the harmful properties that apoA-I lacks. Many substances administered to the lung have a direct pharmacological effect on pneumocytes. They exert this effect in the lung even if undesired, sometimes leading to side effects or toxicity.

The present invention also features the realization that due to the fact that apoA-I is an endogenous protein, there is no danger of an adverse reaction to its administration. Furthermore, apoA-I is non-toxic and inert and has no pharmacological effect on pneumocytes.

According to one embodiment, the present invention features a system and method for determinging the total amount of apoA-I that needs to be delivered through the lung to have a physiological effect. First, the composition of HDL by weight must be considered. Based on existing data, mature HDL contain a ratio of approximately 50:25:1 (phosphotidylcholine:cholesterol:apoA-I) by number. Multiplying by the molecular weight of each, this is equivalent to a ratio of 38:10:26 by weight, which means that apoA-I composes approximately one third of HDL by weight. ApoA-I spontaneously assembles into HDL in the serum, most likely by binding to free and membrane-bound phospholipids. Given that apoA-I is the limiting factor for HDL synthesis in the serum, a given amount of administered apoA-I will result in an increase of the total HDL mass approximately three times the mass of administered apoA-I.

Normal human clearance of HDL is approximately 0.3 pools/day, meaning that approximately ⅓ of the body's HDL is cleared over the course of an entire day. For the body to maintain a relatively constant serum HDL level, the body produces approximately the same amount daily.

To estimate the efficacy of apoA-I delivery, the present invention assumes that 1) the body's rate of HDL production will remain constant, and 2) the body will continue to clear approximately 0.3 pools/day of the total amount of HDL in the serum, clearing endogenously and exogenously derived HDL. Though it may be possible that negative feedback will cause a decrease in the endogenous production of apoA-I, it is the fractional catabolic rate of apoA-I that is dynamic and not the rate of production. Thus, the clearance will increase while the rate of production remains constant.

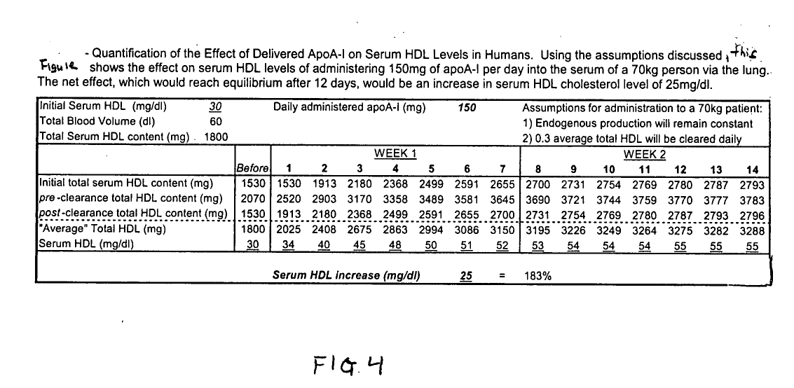

Using these assumptions and the calculations given above, approximately 150 mg of apoA-I administered daily results in an increase in serum HDL cholesterol level of approximately 25 mg/dl, as shown in FIG. 4. For individuals with a serum HDL level as low as approximately 30 mg/dl, this would nearly double the serum HDL level and remove the individuals from a high risk stratum to a low risk stratum.

The present invention includes the realization that delivering approximately 50-200 mg (preferably 150 mg) of apoA-I into the serum through the lung in one day is feasible. Current therapies involving insulin administration, which does not possess many of the physical characteristics that make apoA-I so appropriate for absorption, can deliver 15 mg of insulin into the serum per inhaled breath from a metered dose inhaler. Based on this, 10 breaths from an MDI over the course of a day or a short administration time using a nebulizer can deliver 150 mg of apoA-I to the serum without difficulty.

For perspective, it is interesting to note that in the clinical trail of apoA-I Milano, 15 mg/kg of apoA-I Milano were administered to each individual once per week for five weeks, resulting in the visible regression of atherosclerotic plaques. Given the average 70 kg individual, 15 mg/kg is equal to 1050 mg per week or an average of 150 mg per day, the same amount proposed by the present invention.

The present invention is superior, however, since the pool of apoA-I is almost completely turned over every three days whereas the therapeutic benefit of administrations in the case of apoA-I Milano was absent for more than half of each week. The daily administration of apoA-I via the lung would be active continuously, potentially doubling the clinical efficacy.

The preferred embodiment of the present invention includes a breathable aerosol that contains the target molecule, preferably apoA-I, which is delivered to the peripheral lung and ultimately into the blood stream. The production of such an aerosol is dependent on the formulation to be aerosolized and the aerosolization procedure. The current invention specifies a formulation that meets two criteria.

A first criterion is that the aerosolized formulation should be a stable aerosol that is able to reach the peripheral lung. The peripheral lung is the most highly vascularized and the air-blood barrier is most permeable, thereby allowing for adequate absorption into the blood stream. Inhaled particles stratify to different depths of the bronchial tree depending on their size. The smaller the particles are, the further they can penetrate into the lung. Particles that are too small, however, are exhaled as readily as they are inhaled and little net delivery occurs.

In the preferred embodiment, the therapeutic agent (preferably apoA-I) is aerosolized, using any method or device known to those skilled in the art, to form a heterodispersed aerosol (having a geometric standard deviation greater than approximately 1.2) having an aerodynamic radius of approximately 2 μm to 5 μm, preferably having a mean aerodynamic radius of 3 μm. The size and distribution of the aerosol is preferably measured using a Topas Laser Aerosol Particle Size Spectrometer LAP 320 or the like. This first criterion ensures that the therapeutic formulation reaches the peripheral lung and has access to the circulation system.

The second criterion is that the formulation must contain the therapeutic agent (preferably apoA-I) after being aerosolized. In the preferred embodiment using apoA-I, apoA-I spontaneously assembles with free and cell membrane-associated phospholipids to form HDL. In the serum, the primary agent responsible for reverse cholesterol transport is lipid-poor HDL, which is simply apoA-I associated with a small number of phospholipid molecules. Therefore, aerosolized apoA-I should be delivered to the surface of the lung, so that it may absorb into the serum and self-assemble into HDL.

According to one embodiment, the present invention features a system and method for determining the characteristics of the formulation required to produce a stable aerosol that includes apoA-I and which can reach the peripheral lung. Purified apoA-I (Sigma BCR393) is resolubilized in 6 M guanidine HCL and dialyzed against phosphate buffered saline. The preferred formulation includes a saturated solution of labeled apoA-I in saline. Many of the inhaled therapeutics that are currently administered using nebulizers, such as albuterol, are dissolved in saline. The administration of saline is safe for the lungs and apoA-I will dissolve adequately in water. In the preferred embodiment, apoA-I will be added to saline until the solution is saturated, and the excess will be removed using centrifugation.

In the preferred embodiment, a saturated solution of apoA-I is prepared. Although unlikely, it is possible that the high concentration of protein will make the solution too dense to nebulize. Since saline can easily be nebulized, this variable is tested by adding apoA-I in small increments to saline to determine the maximum concentration.

One skilled in the art will recognize that alternate solvents may also be used. For example, the use of surfactant as a solvent for proteins has been shown to be effective for pulmonary delivery. ApoA-I is known to associate with phospholipids and apoA-I will be nebulized along with the phospholipids. Once inside the lung, the surfactant will simply be added to the existing surfactant, causing no toxicity or complications.

Other solvents may also be used in nebulization, including, but not limited to, benzalkonium chloride, calcium chloride, and sodium citrate. Liposomes or mixed micelles as vectors for pulmonary delivery may also be used, though it is possible that apoA-I delivered in such vectors will not disperse as evenly as free apoA-I.

The present invention features the realization that a dose-loaded suspension may be created by adding the protein to the solvent at a low pH and subsequently mixing vigorously while rapidly raising the pH past the isoelectric point of the protein. This allows the concentration of the protein to be increased past the solubility point.

Once a stable aerosol containing apoA-I is generated, the ability of the protein to enter the blood stream upon delivery to the lung is tested. Ultimately, the concentration of apoA-I in the blood stream that is the important factor; increased serum apoA-I is known to result in increased HDL levels, which has been shown to reverse atherosclerosis.

To facilitate detection of apoA-I in subsequent experiments, lysine residues in molecules of apoA-I will undergo reductive methylation to incorporate a stable isotope, [¹³C], into the molecule. [¹³C]-labeled apoA-I levels can subsequently be detected using NMR at 126 MHz, the optimum frequency for [¹³C]. The presence of [¹³C] in the blood of a test subject is measured before administration of the non-invasive therapy to provide a baseline value. After non-invasive delivery of the therapy, [¹³C] is measured again to determine how much apoA-I has been delivered. This procedure is well documented and has been used previously to detect HDL levels. See Jentoft, N. and Dearborn, D. G. Labeling of proteins by reductive methylation using sodium cyanoborohydride. Journal of Biological Chemistry. 1979; 254 (11):4359-4365, fully incorporated herein by reference.

In the preferred embodiment, using this formulation, or any alternate formulation described herein, an aerosol is generated, preferably using a jet nebulizer. Any other alternative aerosolization apparatus known to those skilled in the art, such as, but not limited to an ultrasonic nebulizer, may also be used.

For a given volume of protein, a sample with a higher surface area will be absorbed faster and more completely. Many agents delivered to the lung aggregate, resulting in large collections of protein with high volume and limited surface contact with the absorptive surface. The consequences of this are a low rate of absorption and the possibility of inflammation and toxicity from large amounts of foreign material obstructing the lung. Because apoA-I disperses into a monolayer, its surface area to volume ratio will be nearly 1.

Furthermore, in addition to absorption by the two standard mechanisms, some of the apoA-I that has integrated into the surfactant will absorb through an additional mechanism. The lung recycles surfactant by absorbing it into Type II pneumocytes; apoA-I integrated in the surfactant will be reabsorbed with the surfactant.

The present invention also features a method to determine whether a formulation, when delivered to the lung, will result in sufficient and/or significant absorption of apoA-I into the blood stream. Furthermore, the relative efficiency of absorption are calculated to allow for comparison between pulmonary and intravenous delivery of apoA-I.

In order to test the ability of the aerosolized formulation to enter the blood stream, three cohorts of mice will be used. The use of mice is commonly used as models for pulmonary delivery studies because the terminal bronchiole diameter of mice is on the same order of magnitude as humans. In the preferred embodiment, neubulized apoA-I will be administered to humans.

Mice are sedated using approximately 0.09 ml per 100 g of body weight of a mixture of approximately 5 ml ketamine (100 mg/ml) and approximately 1.6 ml xylazine (20 mg/ml) and intubated using the FlexiVent system (SCIREQ, Canada). The FlexiVent system is a software controlled ventilator designed for a single mouse. The attached software displays a continuous output of tidal volume, respiratory rate, compliance, resistance, and many other physiological measurements.

The FlexiVent has a built-in nebulizer in series with the ventilator that is specifically designed to deliver aerosols to the peripheral lung. This is accomplished by two mechanisms. During aerosol production, the ventilator will maintain slow, deep breaths to maximize delivery to the peripheral lung. Secondly, the production of aerosol is synchronized with respiration so that aerosols are maximally inhaled. These two modifications along with the measurement of tidal volume provide a relatively accurate estimate of the volume of aerosol delivered to the peripheral lung.

Using this setup, negative controls receive either no formulation, nebulized solvent, or a nebulized solution of solvent and a non-specific protein. The experimental cohort receives a nebulized formulation of [¹³C]-labeled apoA-I as described herein. The positive control cohort is maintained on an identical setup as the other cohorts but receives the same apoA-I formulation as the experimental cohort by intravascular injection instead of by pulmonary delivery. The amount of formulation delivered intravascularly is equal to the estimate of the amount of formulation delivered to the peripheral lung of the experimental cohort, as described above. One of the negative control aerosols may be administered to the positive control mice. Serum concentrations of apoA-I are measured from each cohort at multiple timepoints. The carotid artery is isolated and cannulated to provide blood samples at intervals throughout the experiment.

Approximately 0.1 ml of blood is collected from the cannulated artery and the concentration of [¹³C]-labeled apoA-I is measured using NMR at 126 MHz at various times: for example, but not limited to, before administration, 15 minutes, 30 minutes, 45 minutes, and 1 hour after administration. If the apoA-I concentration has not stabilized by 1 hour, a blood sample is collected every 15 minutes until the concentration plateaus.

In addition to these measurements, the pulmonary function of the subjects is monitored throughout preferably using he Flexivent apparatus. Pulmonary resistance and compliance in conjunction with oxygen saturation is used to monitor for the occurrence of gross pulmonary toxicity.

The statistical significance of the presence of apoA-I is determined by taking multiple measurements of multiple mice and using t-tests to compare the experimental cohort to the negative control cohort. A statistically significant change in the serum levels of apoA-I in experimental cohorts is verifies pulmonary delivery of apoA-I.

In addition to a binary determination of the presence of apoA-I in the blood, the extent and time-course of absorption is analyzed. Measurements of serum apoA-I levels from the positive control cohort yield the desired bioavailability curve. By comparing the bioavailability curve that results from the experimental cohorts to that of the positive control, the efficiency relative to intravenous delivery.

Since the systemic clearance of apoA-I is negligible over the course of the experiment (described below), a comparison of the final concentrations of apoA-I in the blood stream provides the total efficiency. Because absorption through the lung is slower than injection, the timepoints will provide the temporal efficiency of pulmonary absorption.

The metabolism and clearance of apoA-I in the serum will not likely significantly affect the results. The clearance of apoA-I occurs in the kidney, but the fractional catabolic rate is relatively slow. Normal clearance of apoA-I is approximately 0.3 pools/day, meaning that approximately ⅓ of the body's apoA-I is cleared over the course of an entire day. For a very high estimate of clearance, assume that mice clear apoA-I on the same order of magnitude as humans. A calculation can be made of how much apoA-I would be cleared by the kidney within the time period of interest, preferably 60 minutes. Assuming a normal human has an HDL level of approximately 40 mg/dl, a clearance of 0.3 pools/day means that approximately 13 mg/dl is cleared per day or approximately 0.5 mg/dl per hour. In a mouse with a similar clearance rate (though it is most likely considerably less) and a blood volume of 6 ml (6-8% of body weight of a 100 g mouse), the clearance of apoA-I can be estimated to be approximately 0.03 mg in the 60 minute time period, which is negligible.

As further evidence that clearance should not be a problem, in many past experiments during which apoA-I was administered intravenously, HDL was effectively raised and atherosclerosis reduced without any problems with clearance. Though clearance will almost certainly not be a problem, the administration of niacin and fibrates, which reduces the rate of clearance of apoA-I, may be used to increase the half-life of delivered apoA-I in the serum.

The present also includes the realization that the helical portion of apoA-I administered to the serum may be sufficient to augment reverse cholesterol transport. The helical portion of apoA-I can isolated and administered as an aerosol.

In alternative embodiments, absorption may by enhanced by using different formulations described herein such as, but not limited to, using surfactant as a solvent, changing the concentration of apoA-I in solution, or creating a suspension to create an dose-loaded formulation. In addition, alternative permeabilizers such as, but not limited to, ethanol and delivery mechanisms can be used as described herein.

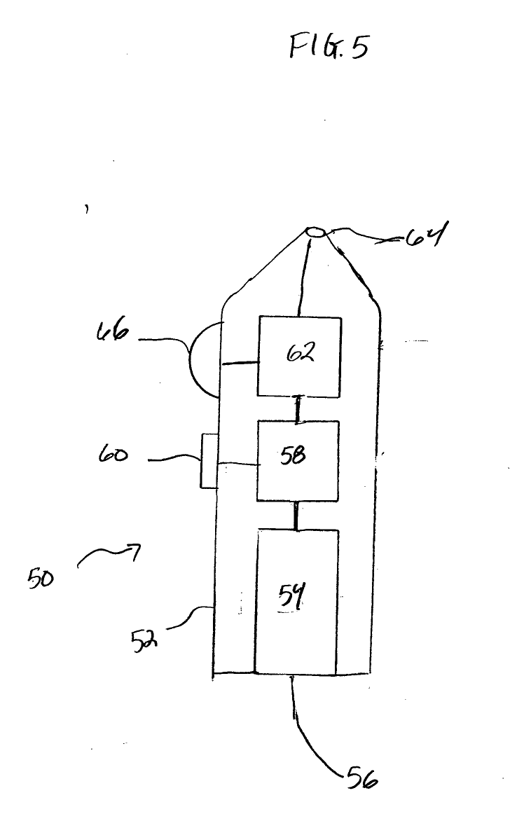

FIG. 5 shows one embodiment of an apparatus 50 for use with the present invention. The apparatus 50 includes a housing 52 and a reservoir 54 containing the solution to be nebulized. The reservoir 54 is preferably sized and shaped to fit at least partially within the housing 52 and may optionally include multiple chambers separating the components of the solution as well as a mixing device (not shown). For example, the reservoir 54 may include a first chamber containing the therapeutic compound (such as apoA-I or any other therapeutic compound disclosed herein) and a second chamber including the solvent, for example the saline solution. The reservoir 54 and/or the housing 52 optionally includes at least one inlet 56 for refilling the reservoir 54.

The apparatus 50 also includes a metering device 58 in fluid communication with the reservoir 54. The metering device 58 measures the dose to be administered, and optionally includes an input device 60 for adjusting the size of the dose.

The metering device 58 is also in fluid communication with an aerosolizing device 62. The aerosolizing device 62 creates a plurality of droplets as is well known to those skilled in the and is in fluid communication with an outlet of nozzle 64 for administering the aerosolized droplets of the solution for each dose. An activation device 66, such as a button or the like, is used to activate the aerosolizing device 62.

Accordingly, the present invention features an apparatus and method for non-invasively treating cardiovascular disease through the use of pulmonary delivery of apolipoproteins and other amphipathic compounds. Because the method and system is non-invasive it is substantially easier and cheaper to administer compared to intravenous therapies. Moreover, the pulmonary delivery of apolipoproteins and other amphipathic compounds can be used to treat other diseases such as, but not limited to, Alzheimer's Disease.

The preferred method of delivery of amphipathic compounds is pulmonary delivery. The requirements for pulmonary delivery are described in detail above, but other non-invasive methods of delivery are equally viable for amphipathic compounds, especially apolipoproteins. Specifically, the formulations of amphipathic proteins described above may be delivered through one of the following methods, including, but not limited to sublingual, nasal, ocular, and transdermal delivery. Certain physiochemical characteristics of amphipathic compounds, specifically apolipoproteins, applies to each of these delivery methods. In particular, each non-invasive method requires the molecule to pass through one or more biological membranes composed of phospholipids. As described in detail above, a crucial physiochemical property of amphipathic compounds, especially apolipoproteins, is that they spontaneously associate with phospholipds, particularly those in biological membranes. The inventor has discovered that the administration of amphipathic molecules, especially apolipoproteins, would readily absorb through these membranes because of this property and a well-documented phenomenon in which molecules associated with a biological membrane spontaneously flip to the opposite site of the membrane. It has been conjectured that this activity is due to the presence of biologically active enzymes called flippases, though the particular activity of this enzyme on apolipoproteins has not been confirmed. As a result of this unique combination of biological features of non-invasive delivery membranes and physiochemical properties of amphipathic molecules, especially apolipoproteins, the following non-invasive delivery methods would be highly effective in the systemic delivery of amphipathic compounds.

To deliver the formulation sublingually, the formulation is administered underneath the tongue and left without swallowing until absorbed. The region under the tongue is a highly vascularized area, and thus would transmit the compound directly into the blood stream. To successfully deliver the formulation sublingually, the formulation is delivered in multiple small doses of approximately 3 ml to maximize the absorptive surface area. Given an approximate concentration of 500 mg/dl, this requires 10 administrations of the formulation beneath the tongue to deliver the desired 150 mg of apoA-I, for example. It is important to note that these concentrations are subject to change as is the desired dosage for other amphipathic compounds or apolipoproteins.

As a highly vascularized area, the inner surface of the nose may also be used for non-invasive delivery of amphipathic compounds. Formulation applied to the inner surface of the nose readily absorb into the blood stream due to the properties of amphipathic compounds, specifically apolipoproteins, discussed above. Liquid drops of formulation are applied to the inner surface of the nose while the patient lies in the supine position. 21 ml drops can be administered to each nostril no sooner than 10 minutes apart. Given an approximate concentration of 500 mg/dl, 30 total drops of the formulation in the nostrils need to be administered to deliver the desired 150 mg of apoA-I, for example. It is important to note that these concentrations are subject to change as is the desired dosage for other amphipathic compounds or apolipoproteins.

Because the eye is also a highly vascularized area, formulation can be administered to the eye using eye drops of approximately 1 ml in each eye no sooner than 5 minutes apart.

Amphipathic compounds, especially apolipoproteins will readily absorb into the blood stream once delivered into the eye for the reasons discussed above. Given an approximate concentration of 500 mg/dl, 30 total drops of the formulation in the eyes need to be administered to deliver the desired 150 mg of apoA-I, for example. It is important to note that these concentrations are subject to change as is the desired dosage for other amphipathic compounds or apolipoproteins.

To deliver the formulation transdermally, a “patch” must be filled with the formulation. The preferred embodiment of a transdermal patch is a reservoir design, though other transdermal patch designs will also be effective with the amphipathic compounds. A reservoir patch includes 5 layers, including an outer backing to protect the drug and adhesive, a “reservoir” of the formulation, a membrane layer through which the amphipathic compound will absorb, an adhesive layer which adheres the patch to the skin, and a liner layer that is peeled of to reveal the adhesive layer. A large reservoir of drug, up to approximately 1 g of apoA-I, for example, may be situated in the reservoir comprising the week's dosage of apoA-I. It is important to note that the quantity of the amphipathic compound in the patch is subject to change for apoA-I and other amphipathic compounds. Additional permeablizers, including, but not limited to ethanol, might be added to the patch to increase absorption through the skin.

As mentioned above, the present invention is not intended to be limited to a system or method which must satisfy one or more of any stated or implied object or feature of the invention and should not be limited to the preferred, exemplary, or primary embodiment(s) described herein. The foregoing description of a preferred embodiment of the invention has been presented for purposes of illustration and description. It is not intended to be exhaustive or to limit the invention to the precise form disclosed. Obvious modifications or variations are possible in light of the above teachings. The embodiment was chosen and described to provide the best illustration of the principles of the invention and its practical application to thereby enable one of ordinary skill in the art to utilize the invention in various embodiments and with various modifications as is suited to the particular use contemplated. All such modifications and variations are within the scope of the invention as determined by the claims when interpreted in accordance with breadth to which they are fairly, legally and equitably entitled.

1. A method for treating a medical condition comprising the act of non-invasively administering a medicament comprising at least one amphipathic compound.

2. The method as claimed in claim 1 wherein said act of non-invasively administering is selected from the group consisting of the acts of pulmonarily administering, transdermally administering, nasally administering, sublingually administering, and ocularly administering.

3. The method as claimed in claim 2 further including the act of preparing said amphipathic compound in a solvent.

4. The method as claimed in claim 3 wherein said solvent includes at least one solvent chosen from the group consisting of saline, surfactant/phospholipids, benzalkonium chloride, calcium chloride, and sodium citrate.

5. The method as claimed in claim 3 wherein said act of suspending said amphipathic compound in said solvent further includes adding said amphipathic compound at a low pH and subsequently mixing vigorously while rapidly raising the pH past the isoelectric point of said amphipathic compound.

6. The method as claimed in claim 3 wherein said amphipathic compound includes at least one apolipoprotein.

7. The method as claimed in claim 6 wherein said at least one apolipoprotein includes at least one apolipoprotein chosen from the group consisting of wild-type apolipoprotein A-I, synthetic apolipoprotein A-I, apolipoprotein E, apolipoprotein E-2, and apolipoprotein J.

8. The method as claimed in claim 6 further including transporting said at least one apolipoprotein into a blood stream to effect therapies directed toward decreasing the extent of atherosclerosis.

9. The method as claimed in claim 8 wherein said apolipoprotein includes at least apolipoprotein A-I (apoA-I).

10. The method as claimed in claim 8 wherein said apolipoprotein includes at least apolipoprotein E.

11. The method as claimed in claim 9 wherein said act of transporting said at least said apoA-I into said blood stream includes transporting approximately 150 mg of apoA-I into said blood stream daily.

12. The method as claimed in claim 9 wherein said apoA-I spontaneously assemblies with free and cell membrane-associated phospholipids to form HDL.

13. The method as claimed in claim 9 wherein said apoA-I includes substantially only the helical portion of apoA-I.

14. The method as claimed in claim 1 wherein said act of non-invasively administering further includes pulmonarily administering an effective amount of said amphipathic compound, wherein said amphipathic compound includes an apolipoprotein prepared in a solvent.

15. The method as claimed in claim 14 wherein said solvent includes at least one solvent chosen from the group consisting of saline, surfactant, benzalkonium chloride, calcium chloride, and sodium citrate.

16. The method as claimed in claim 14 wherein said act of suspending said amphipathic compound in said solvent further includes adding said amphipathic compound at a low pH and subsequently mixing vigorously while rapidly raising the pH past the isoelectric point of said amphipathic compound.

17. The method as claimed in claim 14 wherein said at least one amphipathic compound is less than 200 kDa in size.

18. The method as claimed in claim 14 wherein said act of pulmonarily administering said apolipoprotein further includes forming a substantially heterodispersed aerosol having a hydrodynamic radius of approximately 2 to approximately 5 um.

19. The method as claimed in claim 14 wherein said apolipoprotein includes apolipoprotein A-I.

20. A non-invasive method of delivering amphipathic compound comprising the acts of:
preparing a composition containing at least one amphipathic compound;
aerosolizing said composition to create a plurality of droplets, wherein said plurality of droplets include said at least one amphipathic compound; and
delivering said aerosolized composition to a periphery of a lung, wherein said at least amphipathic compound is transported through an interface of a lung into the blood stream.

21. The non-invasive method as claimed in claim 20 wherein said aerosolizing of said composition is accomplished by a device chosen from the following group consisting of a jet nebulizer, an ultrasonic nebulizer, a dry powder inhaler, a liquid inhaler, a metered dose inhaler, a device that creates an aerosol with inhalable droplets containing said amphipathic compound.

22. The non-invasive method as claimed in claim 20 wherein said act of aerosolizing said composition includes forming a substantially heterodispersed aerosol having a hydrodynamic radius of approximately 2 to approximately 5 um.

23. The non-invasive method as claimed in claim 22 wherein said act of delivering said aerosolized composition to said periphery of said lung further includes forming a substantially even monolayer of said composition proximate said interface of said lung.

24. The non-invasive method as claimed in claim 20 wherein said act of preparing said composition further includes combing said at least one amphipathic compound in a solvent.

25. The non-invasive method as claimed in claim 24 wherein said solvent is chosen from the group consisting of saline, surfactant, benzalkonium chloride, calcium chloride, and sodium citrate.

26. The non-invasive method as claimed in claim 24 wherein said act of preparing said compound further includes adding said amphipathic compound at a low pH and subsequently mixing vigorously while rapidly raising the pH past the isoelectric point of said amphipathic compound.

27. The non-invasive method as claimed in claim 24 wherein said at least one amphipathic compound is less than 200 kDa in size.

28. The non-invasive method as claimed in claim 24 wherein said amphipathic compound includes at least one apolipoprotein.

29. The non-invasive method as claimed in claim 28 wherein said apolipoprotein is chosen from the group consisting of wild-type apolipoprotein A-I, synthetic apolipoprotein A-I, apolipoprotein E, apolipoprotein E-2, and apolipoprotein J.

30. The non-invasive method as claimed in claim 20 wherein said at least one amphipathic compound includes apolipoprotein A-I (apoA-I).

31. The non-invasive method as claimed in claim 30 wherein said act of delivering said aerosolized apoA-I to said periphery of said lung further includes transporting approximately 150 mg of apoA-I into said blood stream daily.

32. The non-invasive method as claimed in claim 30 wherein said apoA-I includes substantially only the helical portion of apolipoprotein A-I.

33. A device for non-invasively delivering amphipathic compound into a blood stream comprising:
a reservoir, said reservoir containing a solution of at least one amphipathic compound in a solvent;
a metering device in fluid communication with said reservoir, said metering device measuring a quantity of said solution to be administered; and
an aerosolizing device, said aerosolizing device creating a plurality of droplets sized to be deliver said aerosolized solution to a periphery of a lung.

38. The device as claimed in claim 33 wherein said amphipathic compound includes at least one apolipoprotein chosen from the group consisting of wild-type apolipoprotein A-I, synthetic apolipoprotein A-I, apolipoprotein E, apolipoprotein E-2, and apolipoprotein J.

39. The device as claimed in claim 33 wherein said solvent includes at least one solvent chosen from the group consisting of saline, surfactant, benzalkonium chloride, calcium chloride, and sodium citrate.
